(19) United States
(12) Patent Application Publication
(10) Pub. No.: US 2005/0142084 A1
(43) Pub. Date: Jun. 30, 2005

(54) Cosmetic compositions containing nacreous pigments of large sized synthetic mica

(76) Inventors: Sanjoy Ganguly, Fairfield, OH (US); Roderic S. Staveley, Mason, OH (US); David L. Dick, Sparta, NJ (US); Stephane R. J. Nicolas, Fleurines (FR); Marguerite Debacker, Brussels (BE); Judith L. Pharo, Hamilton, OH (US); Michael L. Karrick, Middleton, OH (US)

Correspondence Address: Sun Chemical Corporation, 222 Bridge Plaza South, Fort Lee, NJ, 07024, US

(21) Appl. No.: 10/748,017

(22) Filed: Dec. 29, 2003

(51) Int. Cl. A61K 7/021

(52) U.S. Cl. 424/63

(57) ABSTRACT

Cosmetic compositions containing nacreous pigments of large particle size coated synthetic mica and method for increasing or improving the gloss, glint, brightness, whiteness, transparency and compressibility of cosmetic compositions employing same.

### FIELD OF THE INVENTION

This invention relates to cosmetic compositions containing nacreous pigments of large particle sized synthetic mica.

### BACKGROUND OF THE INVENTION

Nacreous pigments, such as micas coated with metal oxides, have been used in cosmetic applications to impart a shiny nacreous or pearlescent effect and have included both natural and synthetic micas.

The commonly used natural mica is muscovite mica, a naturally layered phyllosilicate, representing a monoclinic crystal system composed of sheets of silicate tetrahedrons.

In order to provide interference properties to the natural micas so that they produce a pearlescent or sparkle effect and be used in cosmetic compositions as nacreous pigments, the natural micas are coated with a metal oxides. U.S. Pat. No. 2,278,970 discloses the effect of oxides, such as TiO₂, coated on the mica substrate for use in paint films and coatings. U.S. Pat. No. 3,087,829 describes natural micas coated with various colored metal oxides for use in coatings. However, these coated natural micas lack many of the properties desired for use in cosmetic compositions.

All natural micas possess metallic impurities in between the sheets based on their natural origin. These impurities are known to arise often from substitution of aluminum ions by other metal ions like iron, chromium, zinc etc. The impurities tend to impart dirtiness and a dull look, reducing whiteness, clarity and/or transparency of the natural mica. The metallic impurities also cause internal light absorption which reduces reflectance and transmittance of light through the layers, thereby directly reducing gloss and brightness of the natural mica substrate. Transparency, gloss and brightness are thus negatively affected by such impurities and are therefore problematic for a cosmetic formulator.

For example, various impurities in different amounts affect texture, like coarseness, of the natural mica which may cause variations in the properties of their coated nacreous pigments. Surface irregularities cause diffused reflection and external light scattering, further reducing the brightness and gloss properties of the coated natural micas.

The smoothness of mica surface is known to affect the pearlescent effect as described in U.S. Pat. No. 3,711,308. Surface smoothness also enhances compressibility of any given powdered material such as cosmetic pressed powder. The binding property of pressed powders is dependent on the compressibility of its constituents. The measure of compressibility is often illustrated by the bulk density of a material. A tighter compressibility is indicated by a higher bulk density in grams per cubic centimeter of a powdered material.

For cosmetic composition like pressed powders, blushers and eye makeups, compressibility is an important criterion in their formulations. A nacreous pigment with better compressibility in dry cosmetic systems can minimize or eliminate the use of additional dry binders and fillers. Binders or compressing agents, even when translucent, tend to diminish the desired interference effect of nacreous pigments in a cosmetic composition.

The use of fillers like chalk, talc and mica in pressed powders are used as known in the art. U.S. Pat. No. 4,591,502 discloses non-pearlescent components in pressed powder compositions which reduce the “frosty” effect of the nacreous pigment. U.S. Pat. No. 3,800,034 discloses the use of 40 to 90% amorphous chalk in pressed cosmetic powders. However, as described in U.S. Pat. No. 5,030,446, the drying effect of talc on the skin can sometimes prevent its use in certain pressed powdered materials like compacts, sticks and pellets. The use of natural micas as fillers is therefore quite extensive, but the lack of surface smoothness reduces their compressing ability and the need for suitable quantities of wet and dry binders becomes evident. U.S. Pat. No. 3,978,207 discloses the preparation of cosmetic pressed powders containing 30-90% of nacreous materials such as natural mica coated with TiO₂. The coated natural mica as a filler faces the same challenge of moderate surface smoothness and compressibility as its precursor.

Synthetic mica solved some of the aforementioned visual and texture related problems associated with the coated natural micas. U.S. Pat. No. 5,352,441 describes a powder based lip liner cosmetic composition comprising of a type of talc, low luster pigment, polymers, an emulsifier, and mica filler with particle size between 10-20 microns. U.S. Pat. No. 5,741,355 describes a nacreous pigment of iron oxide coated synthetic mica of around 100 μm particle size where the synthetic mica surface is smoother and has better luster than natural mica. U.S. Pat. No. 5,885,342 describes a pigment, which can be used in cosmetic compositions, containing oxide coated micas having a length of about 1 to 75 microns. U.S. Pat. No. 5,766,577 discloses a powdered color cosmetic composition comprising talc having an average particle size of 2-8 microns (30 to 70 wt. %) and a natural or synthetic uncoated mica having an average particle size of 2-8 microns (30 to 70 wt. %). The mica is added to modify firmness of the cosmetic composition. U.S. Pat. No. 6,056,815 discloses a method for preparing rutile titanium coated platelets where the platelets are either natural or synthetic mica, talc, kaolin or sericite, glass silica flakes or aluminum flakes.

However, the known small particle size synthetic micas, those less than 150 μm in particle size, are still deficient in gloss and brightness properties. The small particle coated synthetic micas (SPCSM), and their natural counterpart, the small particle coated natural mica (SPCNM), have a tendency to show an increase in opacity and reduced transparency which hides much desirable properties such a glint or sparkle of the nacreous pigment. These deficiencies are then carried into cosmetic compositions formulated with or to contain SPCSM and SPCNM.

Thus, there exists a need for cosmetic compositions containing coated synthetic mica having increased transparency, better gloss, glint, brightness and compressibility, while still retaining the desired inherent nacreous pigment purity, such as whiteness.

### SUMMARY OF THE INVENTION

The above objectives of improved transparency, superior gloss, glint and brightness, a better compressibility, and a higher purity indicated by more whiteness, in various different cosmetic compositions can be realized by employing nacreous pigment of a metal oxide coated synthetic mica having a particle size of about 150 μm to about 1000 μm.

The present invention also provides a method of preparing cosmetic compositions containing nacreous pigments described above to increase gloss, glint, whiteness, brightness, transparency and compressibility.

The present invention in particular provides a method of preparing unique cosmetic compositions such as nail polishes, lipsticks, lip-gloss, eye-mascaras, hair-mascaras, loose body powders, talc free body powders, creme to powder eye shadow/blushers, pressed powder eye shadows, pressed powder blushes, hair and body gels, shampoo and body washes, special effects lotions, foundations, eye liners, baby lotions, oils, powders and creams, bath capsules, bath oils, tablets, and salts, bath soaps and detergents, all types of blushers, body and hand preparations, bubble baths, cleansing products like cold creams, cleansing lotions, liquids and pads, colognes and toilet waters, eyebrow pencils, eye lotions, eye makeup preparations, face and neck preparations, face powders, hair shampoos, indoor tanning preparations, leg and body paints, men's talcum, nail extenders, nail enamels, rouges, perfumes, suntan gels, creams and liquids, and the like which contain the nacreous pigments described above.

Other objects and advantages of the present invention will become apparent from the following description and appended claims.

### DETAILED DESCRIPTION OF THE INVENTION

In accordance with the present invention cosmetic compositions having improved transparency, superior gloss, glint and brightness, better compressibility and higher purity indicated by more whiteness, are produced containing nacreous pigments made of large particle size coated synthetic mica (“LPCSM”) wherein the particle size is about 150 μm to about 1000 μm.

The synthetic mica may be a mica such as anadite, annite, aluminoceladonite, aspidolite, bityite, brammallite, biotite, boromuscovite, celadonite, chernykhite, chromphyllite, clintonite, eastonite, ephesite, ferro-aluminoceladonite, ferroceladonite, fuchsite, glauconite, hendricksite, illite, kinoshitalite, lepidolite, margarite, masutomilite, montdorite, muscovite, nanpingite, norrishite, paragonite, phlogopite, polylithionite, preiswerkite, roscoelite, sericite, siderophyllite, tainiolite, tetra-ferri-annite, tetra-ferriphlogopite, tiotite, tobelite, trilithionite, wonesite, zinnwaldite, mixtures thereof and synthetic and artificial mica having a fluoride, chloride, bromide or iodide ion substituted for the hydroxyl group of natural mica as well as baked or calcined products thereof. It is preferred that the synthetic mica be flurophlogopite.

The synthetic mica is coated with a metal oxide or oxides, including but not limited to titanium dioxide, ferric ferrocyanide, chromium oxide, chromium hydroxide or ferric oxide or mixtures thereof. Suitable coatings include but are not limited to titanium dioxides, hydrated oxides of iron (III) and chromium (III), with additional layer including tin (IV) oxide, zirconium (IV) dioxide, ferric ferrocyanide, silicon (IV) dioxide, iron (II) and (III) oxides, aluminum (III) oxide magnesium (II) oxide, chromium (III) oxides, manganese (IV) dioxide and mixtures thereof. It is preferred that the synthetic mica be coated with titanium dioxide and/or iron oxide.

The coated synthetic mica or nacreous pigment has a particle size of about 150 μm to about 1000μ, and more preferably between about 200 μm to about 900 μm, and most preferably between about 200 μm to about 800 μm.

The coated synthetic mica or nacreous pigment is used in cosmetic compositions including, but not limited to nail polishes, lipsticks, lip-gloss, eye-mascaras, hair-mascaras, loose body powders, talc free body powders, creme to powder eye shadow/blushers, pressed powder eye shadows, pressed powder blushes, hair and body gels, shampoo and body washes, special effects lotions, foundations, eye liners, baby lotions, oils, powders and creams, bath capsules, bath oils, tablets, and salts, bath soaps and detergents, all types of blushers, body and hand preparations, bubble baths, cleansing products like cold creams, cleansing lotions, liquids and pads, colognes and toilet waters, eyebrow pencils, eye lotions, eye makeup preparations, face and neck preparations, face powders, hair shampoos, indoor tanning preparations, leg and body paints, men's talcum, nail extenders, nail enamels, rouges, perfumes, suntan gels, creams and liquids, and the like.

The coated synthetic mica or nacreous pigments can be incorporated into formulations utilizing a wide range of materials, at appropriate levels, applicable for creating finished cosmetic formulations. For example, the cosmetic formulations may also include classical pigments such as, but not limited to acid yellow 1, 3 and 73, food red 1 and 17, acid orange 7 and 24, pigment red 4, 57 and 57:1, 63:1 and 65:1, food yellow 3, acid red 23 and 33, food green 3, acid blue 9, food blue 2, solvent red 23, 43, 48, 72 and 73, acid red 87, 92 and 95, solvent yellow 33, acid violet 43, solvent green 3 and 7, solvent violet 13, acid violet 43, vat red 1, including all other organic colors and the approved lakes of all these organic colors as listed in Title 21 of Code of Federal Regulations for Food and Drugs (“CFR”), natural orange 4, yellow 26, white 1, red 4, orange 6 and green 3, pigment metal 1, 2 and 3, blue 29, green 24, white 14, 18, 20, 21 and 25 and pigment green 17 and 18, pigment red 101, yellow 42, black 11, blue 27, violet 16, and white 6 and 4, carmine, bismuth citrate, emerald green, beet powder, cabbage powder, caramel powder, turmeric powder, including all other approved inorganic and natural colors as listed in Title 21 CFR and the International Cosmetic Toiletry and Fragrance Association Handbook (“CTFA”).

The cosmetic compositions of the present invention may also include numerous additives as appropriate for each separate cosmetic application. For example, abrasives, such as kaolin, lauryl acrylate/vinyl pyrrolidone crosspolymer, silica and the like may be included. Also included are absorbents, such as magnesium aluminum silicate, and modified corn starch; anticaking agents, such as alumina, calcium and zinc stearate; antifoaming agents, such as dimethicone, isopropyl alcohol, and petroleum distillates; antioxidants, such as ascorbic acid, butylated hydroxyanisole, butylated hydroxytoluene, and tocopherols; binders, such as acrylates copolymer, carboxymethyl hydroxyethylcellulose, isopropyl palmitate, isostearyl myristate, lanolin alcohol, polybutene, polyethylene; buffering agents, such as sodium and calcium carbonate, diethylamine, sodium bicarbonate, and urea; bulking agents, such as aluminum silicate, calcium sulfate, silk powder, talc, and zinc oxide; chelating agents, such as citric acid, sodium salts of ethylene diamine tetraacetate, and sodium citrate; cosmetic biocides, such as boric acid, coal tar, sulfur, phenol, polyvinyl pyrrolidone-iodine, and zinc acetate; emulsion stabilizers, such as acetylated glycol stearate, cellulose gum, cetyl alcohol, lanolin, lanolin alcohol, polyvinyl pyrrolidone, ozokerite, stearyl alcohol, and polyvinyl acetate; film formers, such acrylate/vinyl pyrrolidone copolymer, corn starch modified, hydroxymethylcellulose, nitrocellulose, polyethylene terephthalate, and polyisobutene; flavoring agents, such as cinnamal, fructose, honey, and vanillin; fragrance components, such as ascorbyl palmitate, citric acid, isododecane, menthol, mineral oil, octyldodecanol, and ricinus communis (castor) seed oil; humectants, such as glycerin, glycols, lactic acid, sorbitol, tripropylene glycol, and urea; opacifying agents, such as hydrated silica, linoleamide, palmitic acid, and titanium dioxide; pH adjusters, such as acetic acid, ammonia, glycolic acid, imidazole, and triethanolamine; plasticizers, such as camphor, dibutyl phthalate, dimethyl adipate, isodecyl citrate, isopropyl citrate, neopentyl glycol, triacetin, and trilauryl phosphate; preservatives, such as butylparaben, diazolidinyl urea, methylparaben, 1,3-bis(hydroxymethyl)-5,5,-dimethylimidiazolidine-2,4-dione, hydantoin, phenoxyethanol, propylparaben, sodium dehydroacetate, and triethanolamide-sorbate; skin-conditioning agents (emollient), such as acetylated lanolin alcohol, cetyl acetate, diisostearyl malate, dimethicone copolyol, hydrogenated palm kernel glycerides, hydrogenated coco-glycerides, isocetyl stearate, mineral oil, and isopropyl myristate; skin-conditioning agents (humectant), such as acetamide monoethanolamide, lactic acid, propylene glycol, and triethanolamide-lactate; skin-conditioning agents (miscellaneous), such as allantoin, coco-betaine, diethanolamide-hydrolyzed lecithin, and dimethicone copolyol acetate; skin-conditioning agents (occlusive), such as acetylated castor oil, caprylic/capric triglyceride, hydrogenated castor oil, lauryl stearate, octyldodecyl ricinoleate, tridecyl trimellitate, trimethylolpropane triisostearate, and trimethylsiloxysilicate; skin protectants, such as aluminum sulfate, lanolin, mineral oil, petrolatum, and talc; slip modifiers, such as magnesium aluminum silicate, magnesium myristate, silk powder, talc, and zinc stearate; solvents, such as butyl acetate, butylene glycol, dibutyl phthalate, diethylhexyl adipate, ethoxyethanol, isobutyl acetate, alcohols, mineral spirits, and water; surface modifiers, such as isopropyl titanium triisostearate, and trimethoxycaprylysilane; surfactants, such as ammonium isostearate, caprylic acid, triethanolamide isostearate, beeswax acid, cetearyl glucoside, deceth-10, dimethicone copolyol adipate, glyceryl ernucate, sodium isostearate, stearic acid, triethanolamide-stearate, cocamidopropyl betaine, cocamide monoethanolamide, cocamine oxide, dicapryl sodium sulfosuccinate, laureth-30, polyoxyehylene castor oil, sodium polystyrene sulfonate, acrylates copolymer, bentonite, vinylpyrrolidone/vinyl acetate copolymer, and silica; viscosity controlling agents, such as glycols, heptane, polyglyceryl sorbiton, turpentine, acetamide monoethanolamide, acrylamide/sodium acrylate copolymer, carbomer, coco-betaine, cocamidopropyl betaine, magnesium aluminum silicate, polyvinyl alcohol, abietyl alcohol, aluminum stearate, beeswax, calcium stearate, ceresin, hydrogenated palm kernel glycerides, isostearyl alcohol, octyldodecyl stearoyl stearate, ozokerite, paraffin, polybutene, synthetic candelilla wax, synthetic carnauba, synthetic wax, and zinc stearate.

In addition to the preferred components enumerated above, others certified for use in cosmetic compositions are within the contemplation of the cosmetic compositions of the present invention as described in the CTFA, which is incorporated herein by reference.

The cosmetic compositions of the present invention may contain between about 0.05 wt. % to about 90 wt. % of nacreous pigment or coated synthetic mica having a particle size of between 150-1000 μm. It is preferred the cosmetic composition contain about 0.25 wt. % to about 75 wt. % of the nacreous pigment.

Depending on the cosmetic composition being formulated, it may contain other additives, such as, between about 0.01 wt. % to about 65 wt. % of classical pigments. Futher, nail polishes may contain between about 0.20 wt. % to about 15 wt. % of abrasives. Loose body powders, nail polishes, pressed powders and creme to powder eye shadows may contain between about 0.50 wt. % to about 75 wt. % of absorbents. Pressed powder eye shadows, pressed powder blushers, eye mascaras, creme to powder eye shadows/blushers and loose powders may contain between about 0.01 wt. % to about 20 wt. % of anticaking agents. Loose body powders, creme to powder eye shadows, pressed powder blushers and eye shadows, lip-glosses, lipsticks and lotions may contain between about 0.01 wt. % to about 7 wt. % of antioxidants. Loose body powders, pressed eye shadows and blushers, creme to powder products may contain between about 0.50 wt. % to about 25 wt. % of binders. Hair-mascaras, shampoos, body washes and lotions may contain between about 0.10 wt. % to about 3 wt. % of buffering agents. Bulking agents may be present between about 0.02 wt. % to about 80 wt. %. Shampoos, body washes, hair and body gels and hair mascaras may contain between about 0.01 wt. % to about 5 wt. % of chelating agents. Eye and hair mascaras, hair and body gels, shampoos, body washes and lotions may contain between about 0.05 wt. % to about 35 wt. % of emulsion stabilizers. Shampoos, body washes, nail polishes, hair and body gels, hair and eye mascaras, creme to powder eye shadows/blushers and lip-glosses may contain between about 0.25 wt. % to about 40 wt. % of film formers. Shampoos and body washes may contain between about 0.01 wt. % to about 12 wt. % of fragrance. Lotions, hair mascaras, lipsticks, hair and body gels may contain between about 0.01 wt. % to about 25 wt. % of humectants. Opacifying agents may be present in about 0.10 wt. % to about 80 wt. %. Shampoos, body washes, hair mascaras, hair and body gels and lotions may contain between about 0.01 wt. % to about 5 wt. % of pH adjusters. Nail polishes may contain between about 0.20 wt. % to about 30 wt. % of plasticizers. Lipsticks, lip-glosses, eye-mascaras, hair-mascaras, loose body powders, creme to powder eye shadows/blushers, pressed powder eye shadows and blushers, hair and body gels, shampoos, body washes and lotions may contain between about 0.01 wt. % to about 6 wt. % of preservatives. Lipsticks, lip-glosses, creme to powder eye shadows/blushers, hair and body gels, shampoos, body washes and lotions may contain between about 0.2 wt. % to about 30 wt. % of skin-conditioning agents. Creme to powder eye shadows/blushers, all dry powders and lotions may contain between about 0.02 wt. % to about 80 wt. % of slip modifiers. Nail polishes, lipsticks, lip-glosses, eye and hair mascaras, creme to powder eye shadows/blushers, hair and body gels, shampoos, body washes and lotions may contain between about 0.1 wt. % to about 99 wt. % of solvents. Eye and hair mascaras, hair and body gels, shampoos, body washes and lotions may contain between about 0.001 wt. % to about 40 wt. % of surfactants. Nail polishes, lipsticks, lip-glosses, eye and hair mascaras, creme to powder eye shadows/blushers, hair and body gels, shampoos, body washes and lotions may contain between about 0.01 wt. % to about 80 wt. % of viscosity controlling agents. Other additives may be present as appropriate for the particular cosmetic composition and are known in the art.

The cosmetic composition of the present invention may be in the form of a powder, stick, pencil, cream, aerosol, liquid, emulsion, dispersion, oil, tablet, capsule, salt, soap, detergent, liner, deodorant and spray, lotion, liquids, pad, tanning, paint, base, enamel, polish, rouge, sachet, lather, gel, pack, tonic, dressing, freshener and the like.

The nacreous pigments contained within the cosmetic composition of the present invention exhibit different visual effects (glint, sparkle and/or interference effects) depending on their illuminating or viewing angles. Large particle sized coated mica, whether natural or synthetic, also scatter light, but the uniformity of the particles as well as their size determines the extent of scattering as particle size in general is inversely proportional to the number of edges. Thus, the large particle size coated natural mica (“LPCNM”) still includes impurities and therefore has reduced transparency, glossiness, whiteness and purity. While not wishing to be bound by theory, it is believed that LPCSM tend to scatter less and have more perceived transparency. It is of importance to have LPCSM with uniform size in order to diminish the opaque look of the SPCSM. The light scattering is much more noticeable for smaller particulate pigments and results in lower transparency/high opacity for SPCSM. The LPCSM however, while preserving its high purity, demonstrates the clear distinction between transparency and whiteness which is due to its reduced scattering. Also, the TiO₂ coating as a percent of the substrate is usually less on the LPCSM which helps boost transparency and clarity. LPCSM appear brighter and glossier than SPCSM. It is also believed that based on their size, LPCSM tend to orient themselves in a flat, horizontal fashion and thereby work as perfect platelets for color interference, maximizing gloss, glint and brightness. The SPCSM may find some difficulty in achieving such uniform orientation and in turn show much reduced properties compared to the LPCSM.

LPCSM having a particle size between 150 μm to about 1000 μm enable the cosmetic formulator to use grades of varied particle size ranges of interference and interference-absorption type coated pigments to their advantage. Thus, cosmetic compositions can be created having superior visual properties such as gloss, glint, transparency, brightness, and whiteness and also enhanced compressibility.

The following examples are not necessarily limited thereto and variations and modifications will be apparent from the disclosure and may be resorted to without departing from the spirit or scope of the invention, as those skilled in the art will readily understand.

### EXAMPLE 1

The cosmetic composition of a nail lacquer illustrating the present invention was prepared from the components set forth in the Table below.

| Components | 1-A | 1-B | 1-C | 1-D | 1-E | 1-F | 1-G | 1-H | 1-I |
|---|---|---|---|---|---|---|---|---|---|
| Nail polish | 94 | 94 | 94 | 94 | 94 | 94 | 94 | 94 | 94 |
| base (Kirker | | | | | | | | | |
| Enterprises, | | | | | | | | | |
| Inc. of | | | | | | | | | |
| Patterson, | | | | | | | | | |
| NJ) | | | | | | | | | |
| Nacreous | 6.0^{(a)} | 6.0^{(b)} | 6.0^{(c)} | 6.0^{(d)} | 6.0^{(e)} | 6.0^{(f)} | 6.0^{(g)} | 6.0^{(h)} | 6.0⁽ⁱ⁾ |
| pigment | | | | | | | | | |
| Total | 100 | 100 | 100 | 100 | 100 | 100 | 100 | 100 | 100 |

^{(a)}white large particle sized synthetic mica coated with TiO₂.
^{(b)}iridescent large particle size synthetic mica coated with TiO₂.
^{(c)}metallic cooper large particle sized synthetic mica coated with iron oxide.
^{(d)}white small particle sized synthetic mica coated with TiO₂
^{(e)}iridescent small particle size synthetic mica coated with TiO₂.
^{(f)}metallic cooper small particle sized synthetic mica coated with iron oxide.
^{(g)}white large particle sized natural mica coated with TiO₂.
^{(h)}iridescent large particle size natural mica coated with TiO₂.
⁽ⁱ⁾metallic cooper large particle sized natural mica coated with iron oxide.

The particle size of the LPCNM and the LPCSM ranged from 150 μm to 750 μm with a mean particle size ranging from 250-360 μm, while the SPCSM has a particle size range of 20-95 μm with a mean particle size ranging from 40-60 μm. The nail lacquers containing the different nacreous pigments were drawn down on a black and white Leneta chart sheet using a 0.0015 Bird applicator and evaluated for whiteness (indication for purity) and also for gloss, brightness, glint and transparency as well as compressibility (which indicates the effect of surface smoothness on bulk density).

Gloss was measured using a BYK Labotron, manufactured by BYK Gardener of Plainview, N.J. at a 60° angle as a percent of specular reflection. Brightness (the numerical value of the reflectance factor of a sample given as a percent) was measured using an X-Rite 938 Spectrodensitometer, manufactured by X-Rite of Grandville, Mich., with the higher percent value denoting better gloss and brightness. Glint was measured by the visual measure of the sparkle reflecting from the nacreous pigment in presence of a light source and rated between 1-5, with 1 being excellent, 2 being very good, 3 being good, 4 being average and 5 being poor. The glint was measured by the same three people for all the evaluations below and in the further Examples. Transparency (reduction in hiding powder) was measured using a X-Rite Multiangle Spectrophotometer, manufactured by X-Rite of Grandville, Mich., at 45° indicating the angle of incidence on the lightness scale L* and measured on the white and black portions of the lacquer coated Leneta card with the higher value denoting a more transparent material. Whiteness (how closely a surface matched the properties of an ideal reflecting surface which neither absorbs nor transmits light but reflects it at equal intensities in all directions) was measured using a Data Color SF600, manufactured by Data Color International, of Lawrencille, N.J., with higher percent value denoting a whiter material. The results of the tests are set forth in the Table below.

| Nacreous | Gloss | Brightness | Glint | Transparency | Whiteness |
|---|---|---|---|---|---|
| Pigments | (%) | (%) | Rating | Index | (%) |
| 1-A | 84 | 67 | 1 | 74 | 66 |
| 1-B | 69 | 69 | 1 | 74 | 65 |
| 1-C | 70 | 27 | 2 | 57 | NM |
| 1-D | 44 | 64 | 2 | 35 | 68 |
| 1-E | 23 | 63 | 3 | 56 | 67 |
| 1-F | 38 | 4 | 4 | 7 | NM |
| 1-G | 58 | 60 | 2 | 67 | 62 |
| 1-H | 50 | 66 | 2 | 68 | 58 |
| 1-I | 60 | 18 | 3 | 50 | NM |

Note:
“NM” denotes that the performance indicator is not measurable in the given application.

### EXAMPLE 2

LPCSM (white, iridescent red, iridescent gold and iridescent green) and LPCNM (white, iridescent red, iridescent gold and iridescent green) were measured for compressibility as indicated by measuring the increase in bulk density as weight per volume. The micas had a particle size of about 150 to 750 μm, with a mean particle size ranging from 250-360 μm. A known weight of each nacreous pigment was placed in a container and shaken using a Thermolyne 37600 Mixer, manufactured by Barnstead/Thermolyne of Dublique, Iowa (precision value of ±5%) and the volume was recorded. The bulk density was calculated as weight per volume (g/cc). A tighter compressibility is indicated by a higher bulk density of the material. The bulk densities were tested three times for each LPCSM and LPCNM and averaged as set forth in the Table below.

| | Bulk Density |
|---|---|
| Nacreous pigment | (g/cc) |
| 2-A (white large particle sized synthetic | 0.510 |
| mica coated with TiO₂.) | |
| 2-B (iridescent red large particle size | 0.554 |
| synthetic mica coated with TiO₂₎ | |
| 2-C (iridescent gold large particle size | 0.629 |
| synthetic mica coated with TiO₂₎ | |
| 2-D (iridescent green large particle size | 0.639 |
| synthetic mica coated with TiO₂₎ | |
| 2-E (white large particle size natural | 0.435 |
| mica coated with TiO₂₎ | |
| 2-F (iridescent red large particle size | 0.497 |
| natural mica coated with TiO₂₎ | |
| 2-G(iridescent gold large particle size | 0.485 |
| natural mica coated with TiO₂₎ | |
| 2-H (iridescent green large particle size | 0.488 |
| natural mica coated with TiO₂₎ | |

### EXAMPLE 3

The cosmetic composition of a nail polish illustrating the present invention was prepared from the components set forth in the Table below.

| Components | 3-A | 3-B | 3-C |
|---|---|---|---|
| Nail polish base | 89.87 | 89.87 | 89.87 |
| D&C Red 6 (barium lake | 2.0 | 2.0 | 2.0 |
| nitrocellulose chip) | | | |
| FD&C Blue 1 (aluminum lake | 0.75 | 0.75 | 0.75 |
| nitroclellulose chip) | | | |
| Metallic copper | 6.38^{(a)} | 6.38^{(b)} | 6.38^{(c)} |
| nacreous pigment | | | |
| (Mica coated with iron oxide) | | | |
| Total | 100 | 100 | 100 |

^{(a)}large particle sized synthetic mica (150-750 μm).
^{(b)}small particle size synthetic mica (20-95 μm).
^{(c)}large particle sized natural mica (150-750 μm).

The barium lake and aluminum lake nitrocellulose pigment chips were dispersed into the nail polish base and mixed well until dispersed and homogenized, being certain not to overheat the mixture. The nacreous pigment was gently added and mixed until uniform. The final product was filled into bottles.

The nail polish compositions were evaluated according to the methods of Example 1 regarding Gloss, Brightness, Glint and Transparency, with the results set forth in the Table below.

| Nacreous | Gloss | Brightness | Glint | Transparency |
|---|---|---|---|---|
| pigment | % | % | Rating | Index |
| 3-A | 69 | 26 | 2 | 57 |
| 3-B | 37 | 4 | 3 | 7 |
| 3-C | 58 | 16 | 3 | 48 |

The numerical evaluation ratings of glint have the following meanings—1 excellent, 2 very good, 3 good, 4 average and 5 poor are a comparative assignment based on visual observations.

### EXAMPLE 4

The cosmetic composition of a moisturizing lipstick illustrating the present invention was prepared from the components set forth in the Table below.

| | Components | 4-A | 4-B | 4-C |
|---|---|---|---|---|
| A | Octyldodecyl ricinoleate | 10.2 | 10.2 | 10.2 |
| B | Castor oil | 18 | 18 | 18 |
| C | Tridecyl trimellitate | 3 | 3 | 3 |
| D | Octyldodecanol | 4 | 4 | 4 |
| E | Tridecyl trimellitate | 3 | 3 | 3 |
| F | Lanolin wax | 6 | 6 | 6 |
| G | Lanolin oil | 6 | 6 | 6 |
| H | Hydrogenated cocoglycerides | 5 | 5 | 5 |
| I | Acetylated lanolin | 3 | 3 | 3 |
| J | Hydrogenated milk glycerides | 5 | 5 | 5 |
| K | Pentaerythritylk tetraisononanoate | 4 | 4 | 4 |
| L | Ozokerite wax | 5 | 5 | 5 |
| M | Candelilla wax | 5 | 5 | 5 |
| N | Carnauba wax | 1 | 1 | 1 |
| O | Synthetic wax | 3 | 3 | 3 |
| P | Butylated hydroxyanisole | 0.5 | 0.5 | 0.5 |
| Q | Propylparaben | 0.15 | 0.15 | 0.15 |
| R | FD&C Yellow 6 (1:2 aluminum | 7.5 | 7.5 | 7.5 |
| | lake castor oil dispersion) | | | |
| S | Black iron oxide castor | 0.6 | 0.6 | 0.6 |
| | oil dispersion (1:2) | | | |
| T | Red iron castor oil dispersion (1:2) | 2 | 2 | 2 |
| U | Metallic gold nacreous pigment (Mica | 8^{(a)} | 8^{(b)} | 8^{(c)} |
| | coated with TiO₂ and Fe₂O₃) | | | |
| | Total | 100 | 100 | 100 |

^{(a)}large particle sized synthetic mica (150-750 μm).
^{(b)}small particle size synthetic mica (20-95 μm).
^{(c)}large particle sized natural mica (150-750 μm).

Components A-O were mixed and heated to 85° C. to melt. Components P and Q were mixed in until fully dispersed. Components R-T were added and the mixture stirred until homogenous. The nacreous pigment (U) was gently blended into the mixture, which was allowed to cool to 55-60° C. and poured into molds, forming the resultant moisturizing lipstick.

### EXAMPLE 5

The cosmetic composition of a lip gloss illustrating the present invention was prepared from the components set forth in the Table below.

| Components | 5-A | 5-B | 5-C |
|---|---|---|---|
| Hydroxystearic acid | 1.46 | 1.46 | 1.46 |
| Trimethylolpropane triisostearate | 10.93 | 10.93 | 10.93 |
| Polybutene | 59 | 59 | 59 |
| Mineral oil | 5.37 | 5.37 | 5.37 |
| Isocetyl stearate | 8.02 | 8.02 | 8.02 |
| Diisostearyl malate | 8.38 | 8.38 | 8.38 |
| FD&C Blue 1 (aluminum lake) | 0.01 | 0.01 | 0.01 |
| D&C Red 7 (calcium lake) | 0.02 | 0.02 | 0.02 |
| Polyethylene tetrephthalate | 0.2 | 0.2 | 0.2 |
| Iridescent blue nacreous pigment | 8^{(a)} | 8^{(b)} | 8^{(c)} |
| (Mica coated with TiO₂) | | | |
| Total | 100 | 100 | 100 |

^{(a)}large particle sized synthetic mica (150-750 μm).
^{(b)}small particle size synthetic mica (20-95 μm).
^{(c)}large particle sized natural mica (150-750 μm).

Hydroxystearic acid and trimethylolpropane triisostearate were mixed at a temperature of 65-75° C. and melted until clear. The pigments were previously pre-mixed in mineral oil and ground to disperse into a color paste then added with the remaining components to the melted mixture, mixing with slow sweeping agitation until uniform. The mixture was cooled to 45° C.

### EXAMPLE 6

The lipsticks of Examples 4 and lip-glosses of Example 5 were evaluated for Gloss, Brightness, Glint, and Transparency by using the visual method and scale of Example 1 (Glint measurement). Whiteness was tested on the lip-glosses only by first melting analogous amounts of finished lip-gloss into paper pans made from the white portion of the Leneta cards and cooled to set and then measured according to the whiteness method of Example 1. The results are set forth in the Table below.

| Nacreous | Gloss | Brightness | Glint | Transparency | Whiteness |
|---|---|---|---|---|---|
| pigment | Rating | Rating | Rating | Rating | % |
| 4-A | 1 | 1 | 1 | 2 | NM |
| 4-B | 2 | 2 | 3 | 4 | NM |
| 4-C | 2 | 3 | 2 | 3 | NM |
| 5-A | 1 | 1 | 1 | 1 | 69 |
| 5-B | 2 | 2 | 3 | 3 | 66 |
| 5-C | 2 | 3 | 3 | 3 | 39 |

Note:
“NM” denotes that the performance indicator is not measurable in the given application. The numerical evaluation ratings of gloss, brightness, glint and transparency having the following meanings, 1 excellent, 2 very good, 3 good, 4 average and 5 poor are a comparative assignment based on visual observations.

### EXAMPLE 7

The cosmetic composition of an eye-mascara illustrating the present invention was prepared from the components set forth in the Table below.

| Components | 7-A | 7-B | 7-C |
|---|---|---|---|
| Petroleum Distillate | 68 | 68 | 68 |
| Polyethylene | 12 | 12 | 12 |
| Dihydroabietyl alcohol | 5 | 5 | 5 |
| Candelilla wax | 2.4 | 2.4 | 2.4 |
| Aluminum stearate | 0.05 | 0.05 | 0.05 |
| Butylparaben | 0.1 | 0.1 | 0.1 |
| Black iron oxide | 4 | 4 | 4 |
| White nacreous | 8^{(a)} | 8^{(b)} | 8^{(c)} |
| pigment (Mica | | | |
| coated with TiO₂) | | | |
| Total | 100 | 100 | 100 |

^{(a)}large particle sized synthetic mica (150-750 μm).
^{(b)}small particle size synthetic mica (20-95 μm).
^{(c)}large particle sized natural mica (150-750 μm).

Petroleum distillate, polyethylene, dihydroabietyl alcohol and candelilla wax were heated to 85-90° C. with medium agitation, until melted. Aluminum stearate and butylparaben were mixed in and then the black iron oxide and white nacreous pigment was added and dispersed at high speed for 45 minutes at 90° C. The mixture was cooled to 40° C.

### EXAMPLE 8

The cosmetic composition of a hair-mascara illustrating the present invention was prepared from the components set forth in the Table below.

| Components | 8-A | 8-B | 8-C |
|---|---|---|---|
| Deionized water | 69.5 | 69.5 | 69.5 |
| Polyvinyl alcohol | 2 | 2 | 2 |
| Propylene glycol | 2 | 2 | 2 |
| Trisodium ethylene diamine | 0.05 | 0.05 | 0.05 |
| tetraacetate | | | |
| Methylparaben | 0.15 | 0.15 | 0.15 |
| Polyvinyl pyrrolidone | 4 | 4 | 4 |
| Triethanolamine | 1 | 1 | 1 |
| Carbomer | 1 | 1 | 1 |
| Diazolidinyl urea | 0.3 | 0.3 | 0.3 |
| White nacreous pigment (Mica | 20^{(a)} | 20^{(b)} | 20^{(c)} |
| coated with TiO₂) | | | |
| Total | 100 | 100 | 100 |

^{(a)}large particle sized synthetic mica (150-750 μm).
^{(b)}small particle size synthetic mica (20-95 μm).
^{(c)}large particle sized natural mica (150-750 μm).

Deionized water (15 grams) is added with polyvinyl alcohol and allowed to completely de-aerate in 4-12 hours, then heated to 60° C. and the propylene glycol, trisodium ethylene diamine tetraacetate, and methylparaben were added. Separately, deionized water (20 grams) and polyvinyl pyrrolidone were combined and then added to the mixture. The mixture was cooled to 45° C. and triethanolamine was added. Separately, deionized water (15 grams) and carbomer were homogenized and then added to the mixture, which was cooled to 45° C. Separately, deionized water (19.5 grams) and diazolidinyl urea were combined and then added to the mixture. The white nacreous pigment was mixed in slowly until homogeneous. The resultant mixture was cooled to 30-35° C. and poured into a mold.

### EXAMPLE 9

The eye-mascaras of Examples 7 and hair-mascaras of Example 8 were evaluated for Gloss, Brightness, Glint, and Transparency by using the visual method and scale of Example 1 (Glint measurement). The results are set forth in the Table below.

| Nacreous | Gloss | Brightness | Glint | Transparency |
|---|---|---|---|---|
| pigment | Rating | Rating | Rating | Rating |
| 7-A | 3 | 2 | 2 | NM |
| 7-B | 4 | 4 | 4 | NM |
| 7-C | 4 | 3 | 4 | NM |
| 8-A | 1 | 1 | 1 | 2 |
| 8-B | 3 | 3 | 4 | 4 |
| 8-C | 2 | 3 | 3 | 3 |

Note:
“NM” denotes that the indicator is not measurable in the given application.
The numerical evaluation ratings of these performance indicators have the following meanings, 1 excellent, 2 very good, 3 good, 4 average and 5 poor are a comparative assignment based on visual observations.

### EXAMPLE 10

The cosmetic composition of a loose body powder illustrating the present invention was prepared from the components set forth in the Table below.

| Components | 10-A | 10-B | 10-C |
|---|---|---|---|
| Talc | 44.5 | 44.5 | 44.5 |
| Calcium stearate | 2 | 2 | 2 |
| Polyethylene | 5 | 5 | 5 |
| Magnesium carbonate | 3 | 3 | 3 |
| Butylated hydroxytoluene | 0.05 | 0.05 | 0.05 |
| Methylparaben | 0.2 | 0.2 | 0.2 |
| Propylparaben | 0.1 | 0.1 | 0.1 |
| Sodium dehydroacetate | 0.1 | 0.1 | 0.1 |
| White nacreous pigment (Mica | 16^{(a)} | 16^{(b)} | 16^{(c)} |
| coated with TiO₂) | | | |
| Iridescent gold nacreous pigment | 12^{(a)} | 12^{(b)} | 12^{(c)} |
| (Mica coated with TiO₂) | | | |
| Metallic gold nacreous pigment | 12^{(a)} | 12^{(b)} | 12^{(c)} |
| (Mica coated with TiO₂ and Fe₂O₃) | | | |
| Trioctanoin | 4.5 | 4.5 | 4.5 |
| Silicone liquid | 0.55 | 0.55 | 0.55 |
| Total | 100 | 100 | 100 |

^{(a)}large particle sized synthetic mica (150-750 μm).
^{(b)}small particle size synthetic mica (20-95 μm).
^{(c)}large particle sized natural mica (150-750 μm).

Talc, calcium stearate, polyethylene, magnesium carbonate, butylated hydroxyanisole, methylparaben, propylparaben, and sodium dehydroacetate were dispersed as a dry blend and mixed with the white, iridescent gold and metallic gold micas. Trioctanoin and silicone liquid were sprayed in and further mixed, resulting in a loose body powder.

### EXAMPLE 11

The cosmetic composition of a talc-free body shimmer loose powder illustrating the present invention was prepared from the components set forth in the Table below.

| Components | 11-A | 11-B | 11-C |
|---|---|---|---|
| Bismuth oxychloride | 30 | 30 | 30 |
| White nacreous pigment (Mica | 40^{(a)} | 40^{(b)} | 40^{(c)} |
| coated with TiO₂) | | | |
| Iridescent gold nacreous pigment | 30^{(a)} | 30^{(b)} | 30^{(c)} |
| (Mica coated with TiO₂) | | | |
| Total | 100 | 100 | 100 |

^{(a)}large particle sized synthetic mica (150-750 μm).
^{(b)}small particle size synthetic mica (20-95 μm).
^{(c)}large particle sized natural mica (150-750 μm).

The components were gently mixed until homogenous, then sieved and taped into containers.

### EXAMPLE 12

The loose body powders of Example 10 and talc-free body shimmer loose powders of Example 11 were evaluated for Gloss, Glint, and Transparency by using the visual method and scale of Example 1 (Glint measurement). Brightness and whiteness were tested by uniformly spreading and tapping known amounts of loose powder into paper pans made from the white portion of Leneta cards and measured according to the method of Example 1. The results are set forth in the Table below.

| Nacreous | Gloss | | Glint | Transparency | Whiteness |
|---|---|---|---|---|---|
| Pigment | Rating | Brightness % | Rating | Rating | % |
| 10-A | 2 | 79 | 2 | 2 | NM |
| 10-B | 4 | 65 | 4 | 4 | NM |
| 10-C | 3 | 72 | 3 | 3 | NM |
| 11-A | 1 | 78 | 1 | 2 | 84 |
| 11-B | 3 | 76 | 4 | 3 | 80 |
| 11-C | 3 | 70 | 3 | 3 | 62 |

Note:
“NM” denotes that the performance indicator is not measurable in the given application. The numerical evaluation ratings of gloss, glint and transparency having the following meanings - 1 excellent, 2 very good, 3 good, 4 average and 5 poor are a comparative assignment based on visual observations.

### EXAMPLE 13

The cosmetic composition of a creme to powder eye shadow/blusher illustrating the present invention was prepared from the components set forth in the Table below.

| | Components | 13-A | 13-B | 13-C |
|---|---|---|---|---|
| A | Ultramarine blue pigment | 0.35 | 0.35 | 0.35 |
| B | Titanium dioxide | 1 | 1 | 1 |
| C | Tan iron oxide | 2 | 2 | 2 |
| D | Brown iron oxide | 0.15 | 0.15 | 0.15 |
| E | Yellow iron oxide | 0.5 | 0.5 | 0.5 |
| F | Isoeicosane | 32 | 32 | 32 |
| G | Isopropyl lanolate | 3.15 | 3.15 | 3.15 |
| H | Octyldodecanol | 4 | 4 | 4 |
| I | Ozokerite | 8.5 | 8.5 | 8.5 |
| J | Silicone fluid | 1.5 | 1.5 | 1.5 |
| K | Jojoba oil | 6.25 | 6.25 | 6.25 |
| L | White nacreous pigment | 16^{(a)} | 16^{(b)} | 16^{(c)} |
| | (Mica coated with TiO₂) | | | |
| M | Polyisobutene | 5 | 5 | 5 |
| N | Hydroxylated lanolin | 3.75 | 3.75 | 3.75 |
| O | Ceresin wax | 3.5 | 3.5 | 3.5 |
| P | Methylparaben | 0.2 | 0.2 | 0.2 |
| Q | Propylparaben | 0.1 | 0.1 | 0.1 |
| R | Butylated hydroxytoluene | 0.05 | 0.05 | 0.05 |
| S | Polymethyl methacrylate | 5 | 5 | 5 |
| T | Silica | 1 | 1 | 1 |
| U | Polyamide | 2 | 2 | 2 |
| V | Iridescent gold nacreous | 20^{(a)} | 20^{(b)} | 20^{(c)} |
| | pigment (Mica coated with | | | |
| | TiO₂) | | | |
| | Total | 100 | 100 | 100 |

^{(a)}large particle sized synthetic mica (150-750 μm).
^{(b)}small particle size synthetic mica (20-95 μm).
^{(c)}large particle sized natural mica (150-750 μm).

Pigments (A-E) were dispersed in isoeicosane fluid (F) and then added with Components G-O and heated to 45-55° C. Components P-R were added and then Components S-V were added incrementally. The mixture was cooled and filled in pans resulting in the creme to powder eye shadow/blusher.

### EXAMPLE 14

The cosmetic composition of a pressed powder eye shadow illustrating the present invention was prepared from the components set forth in the Table below.

| | Components | 14-A | 14-B | 14-C |
|---|---|---|---|---|
| A | Talc | 49.75 | 49.75 | 49.75 |
| B | Titanium dioxide | 1 | 1 | 1 |
| C | Zinc stearate | 5 | 5 | 5 |
| D | Red iron oxide | 0.15 | 0.15 | 0.15 |
| E | Yellow iron oxide | 0.1 | 0.1 | 0.1 |
| F | Polyethylene | 3 | 3 | 3 |
| G | Magnanese violet | 5 | 5 | 5 |
| H | Iridescent red nacreous | 25^{(a)} | 25^{(b)} | 25^{(c)} |
| | pigment (Mica coated with | | | |
| | TiO₂) | | | |
| I | Mineral oil | 7 | 7 | 7 |
| J | Dimethicone fluid | 4 | 4 | 4 |
| | Total | 100 | 100 | 100 |

^{(a)}large particle sized synthetic mica (150-750 μm).
^{(b)}small particle size synthetic mica (20-95 μm).
^{(c)}large particle sized natural mica (150-750 μm).

Components A-G were mixed with iridescent red nacreous pigment (H) and then sprayed with Components I-J The mixture was sifted and pressed into pans applying pressure of about 1000 psi to form a pressed powder eye shadow.

### EXAMPLE 15

The cosmetic composition of a pressed powder blush illustrating the present invention was prepared from the components set forth in the Table below.

| | Components | 15-A | 15-B | 15-C |
|---|---|---|---|---|
| A | Talc | 20 | 20 | 20 |
| B | Titanium dioxide | 5 | 5 | 5 |
| C | Zinc stearate | 7 | 7 | 7 |
| D | Red iron oxide | 2.16 | 2.16 | 2.16 |
| E | Yellow iron oxide | 3.14 | 3.14 | 3.14 |
| F | Polyethylene | 9 | 9 | 9 |
| G | Magnanese violet | 12.2 | 12.2 | 12.2 |
| H | Calcium silicate | 2 | 2 | 2 |
| I | Ultramarine blue | 0.5 | 0.5 | 0.5 |
| J | Methylparaben | 0.2 | 0.2 | 0.2 |
| K | Propylparaben | 0.1 | 0.1 | 0.1 |
| L | Butylated hydroxytoluene | 0.05 | 0.05 | 0.05 |
| M | Sodium dehydroacetate | 0.15 | 0.15 | 0.15 |
| N | White nacreous pigment | 23^{(a)} | 23^{(b)} | 23^{(c)} |
| | (Mica coated with TiO₂) | | | |
| O | Polyamide | 4 | 4 | 4 |
| P | Dimethicone fluid | 3.5 | 3.5 | 3.5 |
| Q | Trioctanoin | 6 | 6 | 6 |
| R | Octyldodecyl stearoyl | 2 | 2 | 2 |
| | stearate | | | |
| S | Trimethylsiloxysilicate | 3.5 | 3.5 | 3.5 |
| | Total | 100 | 100 | 100 |

^{(a)}large particle sized synthetic mica (150-750 μm).
^{(b)}small particle size synthetic mica (20-95 μm).
^{(c)}large particle sized natural mica (150-750 μm).

Components A-M were mixed with the white nacreous pigment (N) and polyamide (O) and then sprayed with Components P-S. The mixture was sifted and pressed into pans applying pressure of about 1000 psi to form a pressed powder blush.

### EXAMPLE 16

The crème to powder eye shadows/blushers of Example 13, the pressed powder eye shadows of Example 14 and the pressed powder blushes of Example 15 were evaluated for Gloss, Brightness, Glint, and Whiteness by using the visual method and scale of Example 1 (Glint measurement). Compressibility was measured by compressing three grams of each powdered sample using a fixed pressure of about 1000 psi with a precision of 50 psi in a Carver Laboratory Press, manufactured by Fred S. Carver Inc., of Menomonee Falls, Wisc. The ratio was determined by using the formula:

$$\frac{y}{x} \times 100$$

wherein x is the smallest height of the compressed powder indicative of maximum compressibility among the six samples, y is the individual height of each compressed sample, with the sample having the smallest height yielding x=y and a compressibility ratio of 100.

A lower number indicates better compressibility. The results are set forth in the Table below

| Nacreous | Gloss | Brightness | Glint | Whiteness | Compressibility |
|---|---|---|---|---|---|
| pigments | Rating | Rating | Rating | Rating | Ratio |
| 13-A | 2 | 1 | 2 | 2 | NM |
| 13-B | 3 | 2 | 4 | 2 | NM |
| 13-C | 4 | 3 | 3 | 4 | NM |
| 14-A | 2 | 1 | 1 | 1 | 118 |
| 14-B | 2 | 1 | 4 | 1 | 118 |
| 14-C | 3 | 3 | 2 | 3 | 122 |
| 15-A | 2 | 1 | 2 | 1 | 105 |
| 15-B | 3 | 2 | 4 | 1 | 100 |
| 15-C | 4 | 3 | 2 | 4 | 111 |

Note:
“NM” denotes that the indicator is not measurable in the given application. The numerical evaluation ratings of these performance indicators namely gloss, brightness, glint and whiteness having the following meanings - 1 excellent, 2 very good, 3 good, 4 average and 5 poor are a comparative assignment based on visual observations.

### EXAMPLE 17

The cosmetic composition of a hair and body gel illustrating the present invention was prepared from the components set forth in the Table below.

| Components | 17-A | 17-B | 17-C |
|---|---|---|---|
| Deionized water | 84 | 84 | 84 |
| Carbomer | 2 | 2 | 2 |
| White nacreous pigment (Mica | 7.8^{(a)} | 7.8^{(b)} | 7.8^{(c)} |
| coated with TiO₂) | | | |
| Glycerin | 2.5 | 2.5 | 2.5 |
| Vinylpyrrolidone/vinyl actetate | 2.5 | 2.5 | 2.5 |
| copolymer | | | |
| Triethanolamine | 1 | 1 | 1 |
| Germaben-11 ®^{(d)} | 0.2 | 0.2 | 0.2 |
| Total | 100 | 100 | 100 |

^{(a)}large particle sized synthetic mica (150-750 μm).
^{(b)}small particle size synthetic mica (20-95 μm).
^{(c)}large particle sized natural mica (150-750 μm).
^{(d)}Manufactured by International Specialty Products of Wayne, NJ; a blend of propylene glycol, diazolindinyl urea, methylparaben and propylparaben.

Carbomer was premixed in deionized water (55 grams) and then the white nacreous pigment and glycerin were added and mixed until homogeneous. The remainder of the components were added and mixed until uniform, resulting in a hair and body gel.

### EXAMPLE 18

The cosmetic composition of a shampoo and body wash illustrating the present invention was prepared from the components set forth in the Table below.

| | Components | 18-A | 18-B | 18-C |
|---|---|---|---|---|
| A | Deionized water | 43.65 | 43.65 | 43.65 |
| B | Cocamide monoethanolamide | 5 | 5 | 5 |
| C | Sodium laureth sulfate | 38 | 38 | 38 |
| D | Cocamidopropyl betaine | 8 | 8 | 8 |
| E | Polyquaternium | 2 | 2 | 2 |
| F | Caustic solution | 1 | 1 | 1 |
| G | Iridescent red nacreous | 1.5^{(a)} | 1.5^{(b)} | 1.5^{(c)} |
| | pigment (Mica coated with | | | |
| | TiO₂) | | | |
| H | Red dye solution (1% sln.) | 0.2 | 0.2 | 0.2 |
| I | Tetrasodium ethylene diamine | 0.1 | 0.1 | 0.1 |
| | tetraacetate | | | |
| J | Germaben-11 ®^{(d)} | 0.5 | 0.5 | 0.5 |
| K | Rose extract | 0.05 | 0.05 | 0.05 |
| | Total | 100 | 100 | 100 |

^{(a)}large particle sized synthetic mica (150-750 μm).
^{(b)}small particle size synthetic mica (20-95 μm).
^{(c)}large particle sized natural mica (150-750 μm).
^{(d)}Manufactured by International Specialty Products of Wayne, NJ; a blend of propylene glycol, diazolindinyl urea, methylparaben and propylparaben.

Components A-F were heated to 75° C., mixed uniformly and the pH adjusted to 6.5. Separately, the iridescent red nacreous pigment (G) and red dye (H) were mixed in until homogeneous and cooled to 40° C. and then added to the mixture. The remaining components were added and mixed in at neutral pH, resulting in a shampoo and body wash.

### EXAMPLE 19

Hair and body gels of Example 17 and the shampoo and body washes of Example 18 were evaluated for Brightness and Glint by using the visual method and scale of Example 1 (Glint measurement). Gloss and whiteness were tested by uniformly spreading known amounts of the gels into paper pans made from the white portion of the Leneta cards and measured according to the method of Example 1. Transparency was tested by uniformly spreading known amounts of the gels into paper pans made from the white and black portions of Leneta cards and measured according to the method of Example 1. The results are set forth in the Table below

| Nacreous | | Brightness | Glint | Transparency | |
|---|---|---|---|---|---|
| pigments | Gloss % | Rating | Rating | Index | Whiteness % |
| 17-A | 10 | 1 | 1 | 17 | 77 |
| 17-B | 5 | 1 | 3 | 11 | 65 |
| 17-C | 6 | 3 | 3 | 13 | 50 |
| 18-A | 32 | 1 | 1 | 53 | 73 |
| 18-B | 26 | 1 | 3 | 33 | 70 |
| 18-C | 22 | 3 | 2 | 42 | 54 |

Note:
The numerical evaluation ratings of brightness and glint having the following meanings - 1 excellent, 2 very good, 3 good, 4 average and 5 poor are a comparative assignment based on visual observations.

### EXAMPLE 20

The cosmetic composition of a special effects lotion illustrating the present invention was prepared from the components set forth in the Table below.

| | Components | 20-A | 20-B | 20-C |
|---|---|---|---|---|
| A | Deionized water | 79.6 | 79.6 | 79.6 |
| B | Carbomer | 0.5 | 0.5 | 0.5 |
| C | Polysorbate | 0.8 | 0.8 | 0.8 |
| D | Propylene glycol | 2 | 2 | 2 |
| E | Glycerin | 5 | 5 | 5 |
| F | Triethanolamine | 0.6 | 0.6 | 0.6 |
| G | Iridescent gold nacreous pigment | 2^{(a)} | 2^{(b)} | 2^{(c)} |
| | (Mica coated with TiO₂) | | | |
| H | Acetylated lanolin alcohol | 3 | 3 | 3 |
| I | Cetyl alcohol | 2 | 2 | 2 |
| J | Stearic acid | 5 | 5 | 5 |
| K | LiquaPar ®^{(d)} | 0.5 | 0.5 | 0.5 |
| | Total | 100.0 | 100 | 100 |

^{(a)}large particle sized synthetic mica (150-750 μm).
^{(b)}small particle size synthetic mica (20-95 μm).
^{(c)}large particle sized natural mica (150-750 μm).
^{(d)}Manufactured by International Specialty Products of Wayne, NJ; a blend of isopropylparaben, isobutylparaben and butylparaben.

Deionized water and carbomer were mixed first and then Components C-G were mixed and heated to 80° C. with moderate agitation. Separately, Components H-J were combined and heated to 85° C. with slow mixing, then added to the mixture and homogenized at 80° C. The mixture was cooled to 50° C. and LiquaPar® was added with slow agitation, resulting in a special effects lotion.

The special effects lotions were evaluated for Gloss, Brightness and Glint by using the visual method and scale of Example 1 (Glint measurement). Whiteness was tested by uniformly spreading known amounts of the lotions into paper pans made from the white portion of Leneta cards and measured according to the method of Example 1. The results are set forth in the Table below

| Nacreous | Gloss | Brightness | Glint | Whiteness |
|---|---|---|---|---|
| pigments | Rating | Rating | Rating | % |
| 20-A | 1 | 1 | 1 | 66 |
| 20-B | 2 | 3 | 4 | 74 |
| 20-C | 3 | 4 | 3 | 57 |

Note:
The numerical evaluation ratings have the following meanings —1 excellent, 2 very good, 3 good, 4 average and 5 poor are a comparative assignment based on visual observations.

The invention has been described in terms of preferred embodiments thereof, but is more broadly applicable as will be understood by those skilled in the art. The invention being thus described, it will be obvious that the same may be varied in many ways. Such variations are not to be regarded as a departure from the spirit and scope of the invention and are intended to be included within the scope of the claims.

1. A cosmetic composition comprising a nacreous pigment having a coated synthetic mica with a particle size ranging from about 150 μm to about 1000 μm.

2. The cosmetic composition of claim 1 wherein the particle size is about 200 μm to about 900 μm.

3. The cosmetic composition of claim 2 wherein the particle size is about 200 μm to about 800 μm.

4. The cosmetic composition of claim 1 wherein the coated synthetic mica has a gloss of at least 40%.

5. The cosmetic composition of claim 4 wherein the coated synthetic mica has a gloss of at least 60%.

6. The cosmetic composition of claim 5 wherein the coated synthetic mica has a gloss of at least 80%.

7. The cosmetic composition of claim 1 having an increased gloss of at least 15% increase.

8. The cosmetic composition of claim 7 wherein the gloss increase is at least 30%.

9. The cosmetic composition of claim 1 wherein the composition has a whiteness of at least 70%.

10. The cosmetic composition of claim 1 wherein the composition has a whiteness of at least 80%.

11. The cosmetic composition of claim 1 having an increased whiteness of at least 5% increase.

12. The cosmetic composition of claim 12 wherein the whiteness increase is at least 10%.

13. The cosmetic composition of claim 1 wherein the composition has a compressibility ratio of less than 120.

14. The cosmetic composition of claim 13 wherein the composition has a compressibility ratio of less than 110.

15. The cosmetic composition of claim 1 wherein the nacreous pigment has a bulk density of at lest 0.5.

16. The cosmetic composition of claim 1 wherein the nacreous pigment has a bulk density of at lest 0.6.

17. The cosmetic composition of claim 1 wherein the nacreous pigment has an increased transparency of at least 10% increase.

18. The cosmetic composition of claim 17 having an increased transparency of at least 15% increase.

19. The cosmetic composition of claim 1 having an increased brightness of at least 3% increase.

20. The cosmetic composition of claim 19 having an increased brightness of at least 5% increase.

21. The cosmetic composition of claim 1 wherein the synthetic mica is selected from the group consisting of muscovite, phlogopite, tiotite, sericite, lepidolite, paragonite, and synthetic mica having at least one fluoride ion substituted for the hydroxyl group of natural mica.

22. The cosmetic composition of claim 21 wherein the synthetic mica is fluorphlogopite.

23. The cosmetic composition of claim 1 wherein the synthetic mica is coated with a metal oxide selected from the group consisting of titanium dioxide, chromium oxide, ferric oxide and a combination thereof.

24. The cosmetic composition of claim 23 wherein the synthetic mica is coated with titanium dioxide.

25. The composition of claim 22 wherein the coated synthetic mica is coated with titanium dioxide.

26. The cosmetic composition of claim 1 wherein the cosmetic composition is selected from the group consisting of nail polishes, lipsticks, lip-gloss, eye-mascaras, hair-mascaras, loose body powders, talc free body powders, creme to powder eye shadow/blushers, pressed powder eye shadows, pressed powder blushes, hair and body gels, shampoo and body washes, special effects lotions, foundations, eye liners, baby lotions, oils, powders and creams, bath capsules, bath oils, tablets, and salts, bath soaps and detergents, all types of blushers, body and hand preparations, bubble baths, cleansing products like cold creams, cleansing lotions, liquids and pads, colognes and toilet waters, eyebrow pencils, eye lotions, eye makeup preparations, face and neck preparations, face powders, hair shampoos, indoor tanning preparations, leg and body paints, men's talcum, nail extenders, nail enamels, rouges, perfumes, suntan gels, creams and liquids.

27. A cosmetic composition having increased gloss comprising the composition of claim 1.

28. A cosmetic composition having increased whiteness comprising the composition of claim 1.

29. A cosmetic composition having increased compressibility comprising the composition of claim 1.

30. A cosmetic composition having increased transparency comprising the composition of claim 1.

31. A cosmetic composition having increased brightness comprising the composition of claim 1.

32. A cosmetic composition having increased glint comprising the composition of claim 1.

33. A method for increasing the gloss of a cosmetic composition comprising adding the composition of claim 1.

34. A method for increasing the whiteness of a cosmetic composition comprising adding the composition of claim 1.

35. A method for increasing the compressibility of a cosmetic composition comprising adding the composition of claim 1.

36. A method for increasing the transparency of a cosmetic composition comprising adding the composition of claim 1.

37. A method for increasing the brightness of a cosmetic composition comprising adding the composition of claim 1.

38. A method for increasing the glint of a cosmetic composition comprising adding the composition of claim 1.
